(19) United States
(12) Patent Application Publication
(10) Pub. No.: US 2024/0400679 A1
(43) Pub. Date: Dec. 5, 2024

(54) ANTI-ROR1 ANTIBODIES AND USES THEREOF

(71) Applicant: IMMUNORIZON LTD., Rehovot (IL)

(72) Inventors: Oren BOGIN, Tel-Aviv (IL); Liat DASSA, Tel Aviv (IL)

(73) Assignee: IMMUNORIZON LTD., Rehovot (IL)

(21) Appl. No.: 18/690,788

(22) PCT Filed: Sep. 11, 2022
(86) PCT No.: PCT/IL2022/050983

§ 371 Date: Mar. 11, 2024

Related U.S. Application Data
(60) Provisional application No. 63/243,150, filed on Sep. 12, 2021.

(51) Int. Cl. C07K 16/28

(52) U.S. Cl. CPC C07K 16/2803; C07K 2317/565

(57) ABSTRACT

The present disclosure describes a number of anti-ROR1 antibodies. Disclosed herein are anti-ROR1 antibodies comprising certain complementarity determining regions (CDRs) as disclosed. The anti-ROR1 antibodies disclosed herein can be used to treat various diseases such as cancer.

### CROSS REFERENCE TO RELATED APPLICATIONS

This application claims the benefit of U.S. Provisional Application Ser. No. 63/243,150, filed on Sep. 12, 2021, which is incorporated in its entirety herein by reference.

### SEQUENCE LISTING STATEMENT

The instant application contains a Sequence Listing which has been submitted electronically in ASCII format and is hereby incorporated by reference in its entirety. Said ASCII copy, created on Aug. 24, 2021, is named P-607766-USP_SL.txt and is 36,797 bytes in size.

### FIELD OF THE INVENTION

The present disclosure relates in general to antibodies. In one embodiment, the present disclosure describes the making and uses of anti-ROR1 antibodies and anti-ROR1 antigen binding fragments thereof.

### BACKGROUND

There has been considerable effort directed at the development of immunotherapeutic approaches for the treatment of cancer; many of which depend on targeting tumor-associated antigens (TAAs). While numerous TAAs have been identified, not all have the appropriate properties to enable safe and effective immune targeting. Such properties include a highly restricted expression profile on normal tissues but broad expression across many different cancer types. Furthermore, cell surface expression is an important property for antibody-targeted therapies.

ROR1 is a member of the receptor tyrosine kinase-like orphan receptor (ROR) family, which belongs to the receptor tyrosine kinase (RTK) family. ROR2 is another member of the ROR family. They are closely related to the tropomyosin-related kinase receptor family (tropomyosin-related kinase, Trk), skeletal muscle specific tyrosine kinase-like receptor family (muscle specific kinase, MuSK) and neurotrophic factor esterine kinase receptor family (NT-RTK).

ROR1 contains two subtypes: complete cell membrane receptor type ROR1 and truncated variants. Truncated variants are mainly divided into two subtypes, membrane-bound ROR1 without extracellular structure and soluble ROR1 with only extracellular structure. There is no difference in the expression of soluble ROR1 between normal people and cancer patients. However, the intact membrane-bound ROR1 is specifically expressed at a high level in a variety of tumor tissues.

ROR1 is a transmembrane receptor tyrosine kinase protein. The structure of ROR1 is highly conserved among biological species, such as human and mouse ROR1 amino acid sequence homology can reach 97%. Human ROR1 consists of an extracellular immunoglobulin-like domain, two cysteine-rich domains (FZD), a proximal membrane kringle domain, and a single transmembrane structure. On the intracellular side, ROR1 possesses an intracellular tyrosine kinase domain (TKD), two serine/threonine enrichment domains (S/TRD) and a proline enrichment domain (PRD).

A series of studies in mice have implicated RORs in skeletal, cardiorespiratory, and neurological development. While ROR1 expression is present during normal embryonic and fetal development, it is absent within most mature tissues. However, the expression of ROR1 has been seen in numerous blood and solid malignancies. This differential expression pattern, low ROR1 expression in adult tissue and high expression in cancer have led investigators to examine the functional advantage conferred to cancer by ROR1 expression and to explore the use of immune-based therapies against ROR1 for targeting cancer cells.

At present, various anti-cancer therapies targeting ROR1, such as monoclonal antibodies, antibody-conjugated drugs (ADC), bispecific antibodies, and CAR-T therapy are being studied and developed. Although ROR1 is a promising immunotherapeutic target in many epithelial tumors, high cell surface ROR1 expression in multiple normal tissues raises concerns for on-target off-tumor toxicities. Thus, clinical translation of ROR1 targeted therapies would require careful monitoring of toxicities to normal organs, and may require strategies to ensure patient safety.

Thus, in view of the potential of targeting ROR1 in anti-cancer therapies, there remains a need to further develop anti-ROR1 antibodies for immunotherapeutic uses.

### SUMMARY

In one embodiment, the present disclosure provides a number of anti-ROR1 antibodies. In one embodiment, each of the anti-ROR1 antibodies comprises three complementarity determining regions (CDRs) on a heavy chain (HCDR1, HCDR2, and HCDR3) and three CDRs on a light chain (LCDR1, LCDR2, and LCDR3), wherein
i. the HCDR1 comprises the amino acid sequences of one of SEQ ID NOs: 15-17, the HCDR2 comprises the amino acid sequences of one of SEQ ID NOs: 18-19, the HCDR3 comprises the amino acid sequences of one of SEQ ID NOs: 20-21, the LCDR1 comprises the amino acid sequences of one of SEQ ID NOs: 22-23, the LCDR2 comprises the amino acid sequences of one of SEQ ID NOs: 24-25, the LCDR3 comprises the amino acid sequences of one of SEQ ID NOs: 26-27; or
ii. the HCDR1 comprises the amino acid sequences of one of SEQ ID NOs: 15, 28, 29, the HCDR2 comprises the amino acid sequences of one of SEQ ID NOs: 30-31, the HCDR3 comprises the amino acid sequences of one of SEQ ID NOs: 32-33, the LCDR1 comprises the amino acid sequences of one of SEQ ID NOs: 34-35, the LCDR2 comprises the amino acid sequences of one of SEQ ID NOs: 36-37, the LCDR3 comprises the amino acid sequences of one of SEQ ID NOs: 38-39; or
iii. the HCDR1 comprises the amino acid sequences of one of SEQ ID NOs: 40-42, the HCDR2 comprises the amino acid sequences of one of SEQ ID NOs: 43-44, the HCDR3 comprises the amino acid sequences of one of SEQ ID NOs: 45-46, the LCDR1 comprises the amino acid sequences of one of SEQ ID NOs: 47-48, the LCDR2 comprises the amino acid sequences of one of SEQ ID NOs: 49-50, the LCDR3 comprises the amino acid sequences of one of SEQ ID NOs: 51-52; or
iv. the HCDR1 comprises the amino acid sequences of one of SEQ ID NOs: 53-55, the HCDR2 comprises the amino acid sequences of one of SEQ ID NOs: 56-57, the HCDR3 comprises the amino acid sequences of one of SEQ ID NOs: 58-59, the LCDR1 comprises the amino acid sequences of one of SEQ ID NOs: 60-61, the LCDR2 comprises the amino acid sequences of one of SEQ ID NOs: 62-63, the LCDR3 comprises the amino acid sequences of one of SEQ ID NOs: 64-65; or
v. the HCDR1 comprises the amino acid sequences of one of SEQ ID NOs: 66-68, the HCDR2 comprises the amino acid sequences of one of SEQ ID NOs: 69-70, the HCDR3 comprises the amino acid sequences of one of SEQ ID NOs: 71-72, the LCDR1 comprises the amino acid sequences of one of SEQ ID NOs: 73-74, the LCDR2 comprises the amino acid sequences of one of SEQ ID NOs: 75-76, the LCDR3 comprises the amino acid sequences of one of SEQ ID NOs: 77-78; or
vi. the HCDR1 comprises the amino acid sequences of one of SEQ ID NOs: 79-81, the HCDR2 comprises the amino acid sequences of one of SEQ ID NOs: 82-83, the HCDR3 comprises the amino acid sequences of one of SEQ ID NOs: 84-85, the LCDR1 comprises the amino acid sequences of one of SEQ ID NOs: 86-87, the LCDR2 comprises the amino acid sequences of one of SEQ ID NOs: 88-89, the LCDR3 comprises the amino acid sequences of one of SEQ ID NOs: 90-91; or
vii. the HCDR1 comprises the amino acid sequences of one of SEQ ID NOs: 40-42, the HCDR2 comprises the amino acid sequences of one of SEQ ID NOs: 92-93, the HCDR3 comprises the amino acid sequences of one of SEQ ID NOs: 94-95, the LCDR1 comprises the amino acid sequences of one of SEQ ID NOs: 96-97, the LCDR2 comprises the amino acid sequences of one of SEQ ID NOs: 98-99, the LCDR3 comprises the amino acid sequences of one of SEQ ID NOs: 51-52.

In one embodiment, each of the anti-ROR1 antibodies comprises a heavy chain variable region and a light chain variable region, wherein the amino acid sequences for the heavy chain variable region and the light chain variable region can be one of the following pairs: SEQ ID NOs: 1 and 2; SEQ ID NOs: 3 and 4; SEQ ID NOs: 5 and 6; SEQ ID NOs: 7 and 8; SEQ ID NOs: 9 and 10; SEQ ID NOs: 11 and 12; or SEQ ID NOs: 13 and 14.

In one embodiment, the present disclosure provides a composition comprising a pharmaceutically acceptable carrier and an anti-ROR1 antibody disclosed herein.

The present disclosure also provides nucleic acid construct comprising one or more nucleic acid sequences that encode a light chain, or a heavy chain, or fragments thereof of the anti-ROR1 antibodies disclosed herein, as well as vectors and host cells comprising such nucleic acid construct.

In one embodiment, the anti-ROR1 antibodies disclosed herein can be used to treat diseases such as cancer, autoimmune diseases, GvHD, viral infection or bacterial infection.

These and other aspects of the anti-ROR1 antibodies will be appreciated from the ensuing descriptions of the figures and detailed description of the anti-ROR1 antibodies.

### DETAILED DESCRIPTION

The present disclosure presents isolated anti-ROR1 antibodies and anti-ROR1 binding domain thereof, wherein unique CDR sequences of anti-ROR1 antibodies are provided within a humanized framework. In addition, the ROR1 antigen binding domain of these anti-ROR1 antibodies can be incorporated into multi-valent antibody construct. The anti-ROR1 antibodies disclosed herein could potentially be used as an immunotherapeutic treatment for a medical condition, for example cancer. The ROR1 antigen used in the development of the anti-ROR1 antibodies and binding domains thereof, as disclosed throughout, was a human ROR1. In some embodiments, the anti-ROR1 antibodies or binding domains thereof bind human ROR1. In some embodiments, the anti-ROR1 antibodies or binding domains thereof bind cynomolgus monkey ROR1.

As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind a ROR1 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

Half maximal effective concentration (EC₅₀) refers to the concentration of a drug, antibody or toxicant which induces a response halfway between the baseline and maximum responses after a specified exposure time. In some embodiments, the response comprises a binding affinity. In some embodiments, the response comprises a functional response for example an agonistic response. A skilled artisan would appreciate that as used herein in certain embodiments, the EC₅₀ measurement of an anti-ROR1 antibody disclosed herein provides a measure of a half-maximal binding of the anti-ROR1 antibody to the ROR1 antigen (EC₅₀ binding). The skilled artisan would appreciate that as used herein in certain embodiments, the EC₅₀ measurement of an anti-ROR1 antibody disclosed herein provides a measure of a half-maximal effective concentration of the anti-ROR1 antibody to induce an agonist response (EC₅₀ functional agonism).

In some embodiments, EC₅₀ comprises the concentration of antibody required to obtain a 50% agonist response that would be observed upon antibody binding. In certain embodiments, a measure of EC₅₀ is commonly used as a measure of a drug's potency and may in some embodiments, reflect the binding of the antibody to the receptor. In some embodiments, anti-ROR1 antibodies having nanomolar EC₅₀ binding concentration measurements comprise tight binding anti-ROR1 antibodies. In some embodiments, anti-ROR1 antibodies having nanomolar EC₅₀ functional agonism concentration measurements comprise functionally effective agonistic antibodies. In certain embodiments, an anti-ROR1 antibody disclosed herein comprises a tight binder to the ROR1 molecule. In certain embodiments, an anti-ROR1 antibody disclosed herein comprises an agonist for the ROR1 molecule. In certain embodiments, an anti-ROR1 antibody disclosed herein comprises a tight binding agonist for the ROR1 molecule. In certain embodiments, an anti-ROR1 antibody disclosed herein comprises an antagonist for the ROR1 molecule. In certain embodiments, an anti-ROR1 antibody disclosed herein comprises a tight binding antagonist for the ROR1 molecule.

In some embodiments, the binding EC₅₀ of an anti-ROR1 antibody is in the nanomolar range. In some embodiments, the binding EC₅₀ of an anti-ROR1 antibody comprises a range of about 0.05-100 nM. In some embodiments, the binding EC₅₀ of an anti-ROR1 antibody comprises a range of about 0.05-50 nM. In some embodiments, the binding binding EC₅₀ of an anti-ROR1 antibody comprises a range of about 0.05-20 nM. In some embodiments, the binding EC₅₀ of an anti-ROR1 antibody comprises a range of about 0.05-10 nM. In some embodiments, the binding EC₅₀ of an anti-ROR1 antibody comprises a range of about 0.1-100 nM. In some embodiments, the binding EC₅₀ of an anti-ROR1 antibody comprises a range of about 0.1-50 nM. In some embodiments, the binding EC₅₀ of an anti-ROR1 antibody comprises a range of about 0.1-20 nM. In some embodiments, the binding EC₅₀ of an anti-ROR1 antibody comprises a range of about 0.1-10 nM. In some embodiments, the binding EC₅₀ of an anti-ROR1 antibody comprises a range of about 1-100 nM. In some embodiments, the binding EC₅₀ of an anti-ROR1 antibody comprises a range of about 1-20 nM. In some embodiments, the binding EC₅₀ of an anti-ROR1 antibody comprises a range of about 20-40 nM. In some embodiments, the binding EC₅₀ of an anti-ROR1 antibody comprises a range of about 40-60 nM. In some embodiments, the binding EC₅₀ of an anti-ROR1 antibody comprises a range of about 60-80 nM. In some embodiments, the binding EC₅₀ of an anti-ROR1 antibody comprises a range of about 80-100 nM. In some embodiments, the binding EC₅₀ of an anti-ROR1 antibody comprises a range of about 1-40 nM. In some embodiments, the binding EC₅₀ of an anti-ROR1 antibody comprises a range of about 1-60 nM. In some embodiments, the binding EC₅₀ of an anti-ROR1 antibody comprises a range of about 1-80 nM. In some embodiments, the binding EC₅₀ of an anti-ROR1 antibody comprises a range of about 1-50 nM. In some embodiments, the binding EC₅₀ of an anti-ROR1 antibody comprises a range of about 0.05-5 nM. In some embodiments, the binding EC₅₀ of an anti-ROR1 antibody comprises a range of about 0.1-5 nM. In some embodiments, the binding EC₅₀ of an anti-ROR1 antibody comprises a range of about 0.05-20 nM.

In some embodiments, the binding EC₅₀ of an anti-ROR1 antibody comprises a range of about 0.05-5 nM. In some embodiments, the binding EC₅₀ of an anti-ROR1 antibody comprises a range of about 0.1-5 nM. In some embodiments, the binding EC₅₀ of an anti-ROR1 antibody comprises a range of about 1-5 nM. In some embodiments, the binding EC₅₀ of an anti-ROR1 antibody comprises a range of about 0.05-10 nM. In some embodiments, the binding EC₅₀ of an anti-ROR1 antibody comprises a range of about 0.1-10 nM. In some embodiments, the binding EC₅₀ of an anti-ROR1 antibody comprises a range of about 1-10 nM. In some embodiments, the binding EC₅₀ of an anti-ROR1 antibody comprises a range of about 5-10 nM. In some embodiments, the binding EC₅₀ of an anti-ROR1 antibody comprises a range of about 0.05-15 nM. In some embodiments, the binding EC₅₀ of an anti-ROR1 antibody comprises a range of about 0.01-15 nM. In some embodiments, the binding EC₅₀ of an anti-ROR1 antibody comprises a range of about 1-15 nM.

In some embodiments, the EC₅₀ measuring functional agonism is referred herein as the function EC₅₀, having all the same qualities. In some embodiments, the functional EC₅₀ of an anti-ROR1 antibody is in the nanomolar range. In some embodiments, the functional EC₅₀ of an anti-ROR1 antibody comprises a range of about 0.05-100 nM. In some embodiments, the functional EC₅₀ of an anti-ROR1 antibody comprises a range of about 0.05-50 nM. In some embodiments, the functional EC₅₀ of an anti-ROR1 antibody comprises a range of about 0.05-20 nM. In some embodiments, the functional EC₅₀ of an anti-ROR1 antibody comprises a range of about 0.05-10 nM. In some embodiments, the functional EC₅₀ of an anti-ROR1 antibody comprises a range of about 0.1-100 nM. In some embodiments, the functional EC₅₀ of an anti-ROR1 antibody comprises a range of about 0.1-50 nM. In some embodiments, the functional EC₅₀ of an anti-ROR1 antibody comprises a range of about 0.1-20 nM. In some embodiments, the functional EC₅₀ of an anti-ROR1 antibody comprises a range of about 0.1-10 nM. In some embodiments, the functional EC₅₀ of an anti-ROR1 antibody comprises a range of about 1-100 nM. In some embodiments, the functional EC₅₀ of an anti-ROR1 antibody comprises a range of about 1-20 nM. In some embodiments, the functional EC₅₀ of an anti-ROR1 antibody comprises a range of about 20-40 nM. In some embodiments, the functional EC₅₀ of an anti-ROR1 antibody comprises a range of about 40-60 nM. In some embodiments, the functional EC₅₀ of an anti-ROR1 antibody comprises a range of about 60-80 nM. In some embodiments, the functional EC₅₀ of an anti-ROR1 antibody comprises a range of about 80-100 nM. In some embodiments, the functional EC₅₀ of an anti-ROR1 antibody comprises a range of about 1-40 nM. In some embodiments, the functional EC₅₀ of an anti-ROR1 antibody comprises a range of about 1-60 nM. In some embodiments, the functional EC₅₀ of an anti-ROR1 antibody comprises a range of about 1-80 nM. In some embodiments, the functional EC₅₀ of an anti-ROR1 antibody comprises a range of about 1-50 nM. In some embodiments, the functional EC₅₀ of an anti-ROR1 antibody comprises a range of about 0.05-5 nM. In some embodiments, the functional EC₅₀ of an anti-ROR1 antibody comprises a range of about 0.1-5 nM. In some embodiments, the functional EC₅₀ of an anti-ROR1 antibody comprises a range of about 0.05-20 nM.

In some embodiments, the functional EC₅₀ of an anti-ROR1 antibody comprises a range of about 0.05-5 nM. In some embodiments, the functional EC₅₀ of an anti-ROR1 antibody comprises a range of about 0.1-5 nM. In some embodiments, the functional EC₅₀ of an anti-ROR1 antibody comprises a range of about 1-5 nM. In some embodiments, the functional EC₅₀ of an anti-ROR1 antibody comprises a range of about 0.05-10 nM. In some embodiments, the functional EC₅₀ of an anti-ROR1 antibody comprises a range of about 0.1-10 nM. In some embodiments, the functional EC₅₀ of an anti-ROR1 antibody comprises a range of about 1-10 nM. In some embodiments, the functional EC₅₀ of an anti-ROR1 antibody comprises a range of about 5-10 nM. In some embodiments, the functional EC₅₀ of an anti-ROR1 antibody comprises a range of about 0.05-15 nM. In some embodiments, the functional EC₅₀ of an anti-ROR1 antibody comprises a range of about 0.01-15 nM. In some embodiments, the functional EC₅₀ of an anti-ROR1 antibody comprises a range of about 1-15 nM.

As used herein, the term “antibody” encompasses an antibody fragment or fragments that retain binding specificity including, but not limited to, IgG, heavy chain variable region (VH), light chain variable region (VL), Fab fragments, F(ab′)₂ fragments, scFv fragments, Fv fragments, a nanobody, minibodies, diabodies, triabodies, tetrabodies, and single domain antibodies (see, e.g., Hudson and Souriau, Nature Med. 9:129-134 (2003)). Also encompassed are humanized, primatized, and chimeric antibodies as these terms are generally understood in the art. In some embodiments, an antibody disclosed herein comprises a precursor construct wherein the antigen binding site may be blocked by a regulatory domain, wherein exposure of the binding site comprise a regulated exposure based on environmental conditions, for example but not limited to exposure to a tumor micro-environment.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen. Thus, the CDR regions are primarily responsible for the specificity of an antigen-binding site. In one embodiment, an antigen-binding site includes six CDRs, comprising the CDRs from each of a heavy and a light chain variable region.

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide. The linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)₂ fragments, scFv fragments, Fv fragments, minibodies, diabodies, triabodies, and tetrabodies.

In one embodiment, the anti-ROR1 antibodies disclosed herein can be incorporated as part of a bispecific antibody. In one embodiment, the anti-ROR1 antibodies disclosed herein can be incorporated as part of a multi-specific antibody. As it is generally known in the art, a bispecific antibody is a recombinant protein that includes antigen-binding fragments of two different monoclonal antibodies, and is thereby capable of binding two different antigens. In one embodiment, the anti-ROR1 antibodies disclosed herein can be incorporated as part of a tri-specific antibody. In one embodiment, the anti-ROR1 antibodies disclosed herein can be incorporated as part of a multi-specific antibody. A multi-specific antibody is a recombinant protein that includes antigen-binding fragments of at least two different monoclonal antibodies, such as two, three or four different monoclonal antibodies. In some embodiments, a multi-specific antibody is a recombinant protein that includes antigen-biding fragment of at least three different monoclonal antibodies. In some embodiments, a multi-specific antibody is a recombinant protein that includes antigen-biding fragment of at least four different monoclonal antibodies.

In some embodiments, the anti-ROR1 antibodies disclosed herein are bi-valent for ROR1. In some embodiments, the anti-ROR1 antibodies disclosed herein are monovalent for binding ROR1.

In some embodiments, bispecific, tri-specific, or multi-specific antibodies are used for cancer immunotherapy by simultaneously targeting more than one antigen target, for example but not limited to, a cytotoxic T cell (CTL) and a tumor associated antigen (TAA), or a T cell and a TAA, or a natural killer (NK) cell and a TAA, or simultaneously targeting a T cell, a NK cell, and a TAA. The TAA can be, but is not limited to, ROR1.

### Anti-ROR1 Antibodies

The present disclosure provides a number of anti-ROR1 antibodies. In one embodiment, each of the anti-ROR1 antibodies comprises a set of three complementarity determining regions (CDRs) on a heavy chain (HCDR1, HCDR2, and HCDR3) and a set of three CDRs on a light chain (LCDR1, LCDR2, and LCDR3), wherein
i. the HCDR1 comprises the amino acid sequences of one of SEQ ID NOs: 15-17, the HCDR2 comprises the amino acid sequences of one of SEQ ID NOs: 18-19, the HCDR3 comprises the amino acid sequences of one of SEQ ID NOs: 20-21, the LCDR1 comprises the amino acid sequences of one of SEQ ID NOs: 22-23, the LCDR2 comprises the amino acid sequences of one of SEQ ID NOs: 24-25, the LCDR3 comprises the amino acid sequences of one of SEQ ID NOs: 26-27; or
ii. the HCDR1 comprises the amino acid sequences of one of SEQ ID NOs: 15, 28, 29, the HCDR2 comprises the amino acid sequences of one of SEQ ID NOs: 30-31, the HCDR3 comprises the amino acid sequences of one of SEQ ID NOs: 32-33, the LCDR1 comprises the amino acid sequences of one of SEQ ID NOs: 34-35, the LCDR2 comprises the amino acid sequences of one of SEQ ID NOs: 36-37, the LCDR3 comprises the amino acid sequences of one of SEQ ID NOs: 38-39; or
iii. the HCDR1 comprises the amino acid sequences of one of SEQ ID NOs: 40-42, the HCDR2 comprises the amino acid sequences of one of SEQ ID NOs: 43-44, the HCDR3 comprises the amino acid sequences of one of SEQ ID NOs: 45-46, the LCDR1 comprises the amino acid sequences of one of SEQ ID NOs: 47-48, the LCDR2 comprises the amino acid sequences of one of SEQ ID NOs: 49-50, the LCDR3 comprises the amino acid sequences of one of SEQ ID NOs: 51-52; or
iv. the HCDR1 comprises the amino acid sequences of one of SEQ ID NOs: 53-55, the HCDR2 comprises the amino acid sequences of one of SEQ ID NOs: 56-57, the HCDR3 comprises the amino acid sequences of one of SEQ ID NOs: 58-59, the LCDR1 comprises the amino acid sequences of one of SEQ ID NOs: 60-61, the LCDR2 comprises the amino acid sequences of one of SEQ ID NOs: 62-63, the LCDR3 comprises the amino acid sequences of one of SEQ ID NOs: 64-65; or
v. the HCDR1 comprises the amino acid sequences of one of SEQ ID NOs: 66-68, the HCDR2 comprises the amino acid sequences of one of SEQ ID NOs: 69-70, the HCDR3 comprises the amino acid sequences of one of SEQ ID NOs: 71-72, the LCDR1 comprises the amino acid sequences of one of SEQ ID NOs: 73-74, the LCDR2 comprises the amino acid sequences of one of SEQ ID NOs: 75-76, the LCDR3 comprises the amino acid sequences of one of SEQ ID NOs: 77-78; or
viii. the HCDR1 comprises the amino acid sequences of one of SEQ ID NOs: 79-81, the HCDR2 comprises the amino acid sequences of one of SEQ ID NOs: 82-83, the HCDR3 comprises the amino acid sequences of one of SEQ ID NOs: 84-85, the LCDR1 comprises the amino acid sequences of one of SEQ ID NOs: 86-87, the LCDR2 comprises the amino acid sequences of one of SEQ ID NOs: 88-89, the LCDR3 comprises the amino acid sequences of one of SEQ ID NOs: 90-91: or
ix. the HCDR1 comprises the amino acid sequences of one of SEQ ID NOs: 40-42, the HCDR2 comprises the amino acid sequences of one of SEQ ID NOs: 92-93, the HCDR3 comprises the amino acid sequences of one of SEQ ID NOs: 94-95, the LCDR1 comprises the amino acid sequences of one of SEQ ID NOs: 96-97, the LCDR2 comprises the amino acid sequences of one of SEQ ID NOs: 98-99, the LCDR3 comprises the amino acid sequences of one of SEQ ID NOs: 51-52.

In another embodiment, the anti-ROR1 antibodies comprises heavy chain and light chain CDR sequences that are at least 80% (e.g., at least 85%, 90%, 95%, 96%, 97%, 98%, or 99%) identical to the amino acid sequences set forth above, for example but not limited to as determined using BlastP software of the National Center of Biotechnology Information (NCBI) using default parameters.

One skilled in the art would appreciate that the term percent homology may encompass % identity between two sequences (e.g., between polypeptide sequences or between nucleotide sequences). As used throughout this application, in certain embodiments, ROR1 antibodies or binding fragments thereof, may have substantial identity to a polypeptide sequence of a ROR1 antibody or binding domain thereof as described herein. For example, a ROR1 antibody may comprise a polypeptide comprising at least 70% sequence identity, preferably at least 75%, 80%, 85%, 90%, 95%, 96%, 97%, 98%, or 99% or higher, sequence identity to a reference polypeptide sequence such as a sequence of a ROR1 antibody or domain thereof described herein, using the methods generally known in the art (e.g., BLAST analysis using standard parameters).

In one embodiment, each of the anti-ROR1 antibodies presented herein comprises a heavy chain variable region (VH) and a light chain variable region (VL), wherein the amino acid sequences for the heavy chain variable region and the light chain variable region can be one of the following pairs: SEQ ID NOs: 1 and 2; SEQ ID NOs: 3 and 4; SEQ ID NOs: 5 and 6; SEQ ID NOs: 7 and 8; SEQ ID NOs: 9 and 10; SEQ ID NOs: 11 and 12; or SEQ ID NOs: 13 and 14.

In another embodiment, the anti-ROR1 antibodies comprise VH and VL sequences that are at least 80% (e.g., at least 85%, 90%, 95%, 96%, 97%, 98%, or 99%) identical to the amino acid sequences set forth above, for example but not limited to as determined using BlastP software of the National Center of Biotechnology Information (NCBI) using default parameters.

In view of the sequences for the heavy chain variable regions and light chain variable regions disclosed herein, one of ordinary skill in the art would readily employ standard techniques known in the art to construct an anti-ROR1 scFv.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In another embodiment, the VH and VL domains may be combined in a suitable buffer containing an enzyme and/or a cofactor which can promote dimerization of the VH and VL domains. In another embodiment, the VH and VL domains may be combined in a suitable vehicle that allows them to react with each other in the presence of a suitable reagent and/or catalyst.

In certain embodiments, the VH and VL domains may be contained within longer polypeptide sequences that may include for example, but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g., CH1, CH2, CH3, CH4, Ck, Cl). In some embodiments, the longer polypeptides may comprise multiple copies of one or both of the VH and VL domains generated according to the method disclosed herein; for example, when the polypeptides generated herein are used to forms a diabody a tribody or any multi-armed antibodies.

In one embodiment, the anti-ROR1 antibody presented herein can be an IgG, a Fv, a scFv, a Fab, a F(ab′)₂, a minibody, a diabody, a tribody, a nanobody, a bispecific antibody, tri-specific, multi-specific, or a single domain antibody. For example, the anti-ROR1 antibody can be IgG such as IgG1, IgG2, IgG3, or lgG4.

In one embodiment, the present disclosure provides antibodies that bind with high affinity to ROR1. In one embodiment, binding affinity is calculated by a modification of the Scatchard method as described by Frankel et al. (Mol. Immunol., 16:101-106, 1979). In another embodiment, binding affinity is measured by an antigen/antibody dissociation rate. In another embodiment, binding affinity is measured by a competition radioimmunoassay. In another embodiment, binding affinity is measured by ELISA. In another embodiment, antibody affinity is measured by flow cytometry.

In one embodiment, the present disclosure also provides nucleic acid constructs comprising one or more nucleic acid sequences that encode the anti-ROR1 antibodies disclosed herein, wherein the nucleic acid sequences encode anti-ROR1 antibodies comprising a set of three complementarity determining regions (CDRs) on a heavy chain (HCDR1, HCDR2, and HCDR3) and a set of three CDRs on a light chain (LCDR1, LCDR2, and LCDR3), wherein the HCDR1 comprises the amino acid sequences of one of SEQ ID NOs: 15-17, the HCDR2 comprises the amino acid sequences of one of SEQ ID NOs: 18-19, the HCDR3 comprises the amino acid sequences of one of SEQ ID NOs: 20-21, the LCDR1 comprises the amino acid sequences of one of SEQ ID NOs: 22-23, the LCDR2 comprises the amino acid sequences of one of SEQ ID NOs: 24-25, the LCDR3 comprises the amino acid sequences of one of SEQ ID NOs: 26-27.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a set of three complementarity determining regions (CDRs) on a heavy chain (HCDR1, HCDR2, and HCDR3) and a set of three CDRs on a light chain (LCDR1, LCDR2, and LCDR3), wherein the HCDR1 comprises the amino acid sequences of one of SEQ ID NOs: 15, 28, 29, the HCDR2 comprises the amino acid sequences of one of SEQ ID NOs: 30-31, the HCDR3 comprises the amino acid sequences of one of SEQ ID NOs: 32-33, the LCDR1 comprises the amino acid sequences of one of SEQ ID NOs: 34-35, the LCDR2 comprises the amino acid sequences of one of SEQ ID NOs: 36-37, the LCDR3 comprises the amino acid sequences of one of SEQ ID NOs: 38-39.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a set of three complementarity determining regions (CDRs) on a heavy chain (HCDR1, HCDR2, and HCDR3) and a set of three CDRs on a light chain (LCDR1, LCDR2, and LCDR3), wherein the HCDR1 comprises the amino acid sequences of one of SEQ ID NOs: 40-42, the HCDR2 comprises the amino acid sequences of one of SEQ ID NOs: 43-44, the HCDR3 comprises the amino acid sequences of one of SEQ ID NOs: 45-46, the LCDR1 comprises the amino acid sequences of one of SEQ ID NOs: 47-48, the LCDR2 comprises the amino acid sequences of one of SEQ ID NOs: 49-50, the LCDR3 comprises the amino acid sequences of one of SEQ ID NOs: 51-52.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a set of three complementarity determining regions (CDRs) on a heavy chain (HCDR1, HCDR2, and HCDR3) and a set of three CDRs on a light chain (LCDR1, LCDR2, and LCDR3), wherein the HCDR1 comprises the amino acid sequences of one of SEQ ID NOs: 53-55, the HCDR2 comprises the amino acid sequences of one of SEQ ID NOs: 56-57, the HCDR3 comprises the amino acid sequences of one of SEQ ID NOs: 58-59, the LCDR1 comprises the amino acid sequences of one of SEQ ID NOs: 60-61, the LCDR2 comprises the amino acid sequences of one of SEQ ID NOs: 62-63, the LCDR3 comprises the amino acid sequences of one of SEQ ID NOs: 64-65.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a set of three complementarity determining regions (CDRs) on a heavy chain (HCDR1, HCDR2, and HCDR3) and a set of three CDRs on a light chain (LCDR1, LCDR2, and LCDR3), wherein the HCDR1 comprises the amino acid sequences of one of SEQ ID NOs: 66-68, the HCDR2 comprises the amino acid sequences of one of SEQ ID NOs: 69-70, the HCDR3 comprises the amino acid sequences of one of SEQ ID NOs: 71-72, the LCDR1 comprises the amino acid sequences of one of SEQ ID NOs: 73-74, the LCDR2 comprises the amino acid sequences of one of SEQ ID NOs: 75-76, the LCDR3 comprises the amino acid sequences of one of SEQ ID NOs: 77-78.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a set of three complementarity determining regions (CDRs) on a heavy chain (HCDR1, HCDR2, and HCDR3) and a set of three CDRs on a light chain (LCDR1, LCDR2, and LCDR3), wherein the HCDR1 comprises the amino acid sequences of one of SEQ ID NOs: 79-81, the HCDR2 comprises the amino acid sequences of one of SEQ ID NOs: 82-83, the HCDR3 comprises the amino acid sequences of one of SEQ ID NOs: 84-85, the LCDR1 comprises the amino acid sequences of one of SEQ ID NOs: 86-87, the LCDR2 comprises the amino acid sequences of one of SEQ ID NOs: 88-89, the LCDR3 comprises the amino acid sequences of one of SEQ ID NOs: 90-91.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a set of three complementarity determining regions (CDRs) on a heavy chain (HCDR1, HCDR2, and HCDR3) and a set of three CDRs on a light chain (LCDR1, LCDR2, and LCDR3), wherein the HCDR1 comprises the amino acid sequences of one of SEQ ID NOs: 40-42, the HCDR2 comprises the amino acid sequences of one of SEQ ID NOs: 92-93, the HCDR3 comprises the amino acid sequences of one of SEQ ID NOs: 94-95, the LCDR1 comprises the amino acid sequences of one of SEQ ID NOs: 96-97, the LCDR2 comprises the amino acid sequences of one of SEQ ID NOs: 98-99, the LCDR3 comprises the amino acid sequences of one of SEQ ID NOs: 51-52.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a heavy chain variable region and a light chain variable region, wherein the amino acid sequences for the heavy chain variable region and the light chain variable region have the sequences of SEQ ID NOs: 1 and 2.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a heavy chain variable region and a light chain variable region, wherein the amino acid sequences for the heavy chain variable region and the light chain variable region have the sequences of SEQ ID NOs: 3 and 4.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a heavy chain variable region and a light chain variable region, wherein the amino acid sequences for the heavy chain variable region and the light chain variable region have the sequences of SEQ ID NOs: 5 and 6.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a heavy chain variable region and a light chain variable region, wherein the amino acid sequences for the heavy chain variable region and the light chain variable region have the sequences of SEQ ID NOs: 7 and 8.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a heavy chain variable region and a light chain variable region, wherein the amino acid sequences for the heavy chain variable region and the light chain variable region have the sequences of SEQ ID NOs: 9 and 10.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a heavy chain variable region and a light chain variable region, wherein the amino acid sequences for the heavy chain variable region and the light chain variable region have the sequences of SEQ ID NOs: 11 and 12.

In one embodiment, the present disclosure provides nucleic acid constructs comprising one or more nucleic acid sequences that encode the anti-ROR1 antibodies disclosed herein, wherein the nucleic acid sequences encode anti-ROR1 antibodies comprising a heavy chain variable region and a light chain variable region, wherein the amino acid sequences for the heavy chain variable region and the light chain variable region have the sequences of SEQ ID NOs: 13 and 14.

In another embodiment, the present disclosure also provides a vector comprising the nucleic acid constructs described above. In view of the amino acid sequences disclosed herein, one of ordinary skill in the art would readily construct a vector or plasmid to encode for the amino acid sequences, or homologs thereof. In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

### Compositions for Use

In one embodiment, the present disclosure provides a composition comprising the anti-ROR1 antibody disclosed herein and a pharmaceutically acceptable carrier. Pharmaceutically acceptable carriers of use are well-known in the art. For example, Remington's Pharmaceutical Sciences, by E. W. Martin, Mack Publishing Co., Easton, PA, 15th Edition, 1975, describes compositions and formulations suitable for pharmaceutical delivery of the antibodies disclosed herein. In one embodiment, the composition comprises anti-ROR1 antibodies that comprise a set of three complementarity determining regions (CDRs) on a heavy chain (HCDR1, HCDR2, and HCDR3) and a set of three CDRs on a light chain (LCDR1, LCDR2, and LCDR3) as disclosed herein.

In other embodiments, the composition comprises anti-ROR1 antibodies having heavy chain and light chain CDR sequences that are at least 80% (e.g., at least 85%, 90%, 95%, 96%, 97%, 98%, or 99%) identical to the amino acid sequences set forth above.

In another embodiment, the composition comprises anti-ROR1 antibodies having one of the pairs of heavy chain variable region (VH) and light chain variable region (VL) as disclosed herein.

In another embodiment, the composition comprises anti-ROR1 antibodies having VH and VL sequences that are at least 80% (e.g., at least 85%, 90%, 95%, 96%, 97%, 98%, or 99%) identical to the amino acid sequences set forth above, for example but not limited to as determined using BlastP software of the National Center of Biotechnology Information (NCBI) using default parameters.

In some embodiments of compositions, the antibodies disclosed herein can be in the form of a conjugate. As used herein, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, carbohydrate or recombinant virus. An antibody conjugate can also be referred to as an “immunoconjugate.” When the conjugate comprises an antibody linked to a drug (e.g., a cytotoxic agent), the conjugate can be referred to as an “antibody-drug conjugate”. Other antibody conjugates include, for example, multi-specific (such as bispecific or trispecific or tetraspecific) antibodies and chimeric antigen receptors (CARs).

A composition comprising the anti-ROR1 antibody or an antigen-binding fragment thereof can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodology well known in the pharmaceutical art.

The pharmaceutical compositions comprising the antibodies or antigen-binding fragments thereof disclosed herein can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Alternatively, administration can be intratumoral, e.g., local or intravenous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

### Methods of Use

The present disclosure also provides methods of using the anti-ROR1 antibodies comprising three complementarity determining regions (CDRs) on a heavy chain (HCDR1, HCDR2, and HCDR3) and three CDRs on a light chain (LCDR1, LCDR2, and LCDR3), wherein the HCDR1 comprises the amino acid sequences of one of SEQ ID NOs: 15-17, the HCDR2 comprises the amino acid sequences of one of SEQ ID NOs: 18-19, the HCDR3 comprises the amino acid sequences of one of SEQ ID NOs: 20-21, the LCDR1 comprises the amino acid sequences of one of SEQ ID NOs: 22-23, the LCDR2 comprises the amino acid sequences of one of SEQ ID NOs: 24-25, the LCDR3 comprises the amino acid sequences of one of SEQ ID NOs: 26-27.

The present disclosure also provides methods of using the anti-ROR1 antibodies comprising three complementarity determining regions (CDRs) on a heavy chain (HCDR1, HCDR2, and HCDR3) and three CDRs on a light chain (LCDR1, LCDR2, and LCDR3), wherein the HCDR1 comprises the amino acid sequences of one of SEQ ID NOs: 15, 28, 29, the HCDR2 comprises the amino acid sequences of one of SEQ ID NOs: 30-31, the HCDR3 comprises the amino acid sequences of one of SEQ ID NOs: 32-33, the LCDR1 comprises the amino acid sequences of one of SEQ ID NOs: 34-35, the LCDR2 comprises the amino acid sequences of one of SEQ ID NOs: 36-37, the LCDR3 comprises the amino acid sequences of one of SEQ ID NOs: 38-39.

The present disclosure also provides methods of using the anti-ROR1 antibodies comprising three complementarity determining regions (CDRs) on a heavy chain (HCDR1, HCDR2, and HCDR3) and three CDRs on a light chain (LCDR1, LCDR2, and LCDR3), wherein the HCDR1 comprises the amino acid sequences of one of SEQ ID NOs: 40-42, the HCDR2 comprises the amino acid sequences of one of SEQ ID NOs: 43-44, the HCDR3 comprises the amino acid sequences of one of SEQ ID NOs: 45-46, the LCDR1 comprises the amino acid sequences of one of SEQ ID NOs: 47-48, the LCDR2 comprises the amino acid sequences of one of SEQ ID NOs: 49-50, the LCDR3 comprises the amino acid sequences of one of SEQ ID NOs: 51-52.

The present disclosure also provides methods of using the anti-ROR1 antibodies comprising three complementarity determining regions (CDRs) on a heavy chain (HCDR1, HCDR2, and HCDR3) and three CDRs on a light chain (LCDR1, LCDR2, and LCDR3), wherein the HCDR1 comprises the amino acid sequences of one of SEQ ID NOs: 53-55, the HCDR2 comprises the amino acid sequences of one of SEQ ID NOs: 56-57, the HCDR3 comprises the amino acid sequences of one of SEQ ID NOs: 58-59, the LCDR1 comprises the amino acid sequences of one of SEQ ID NOs: 60-61, the LCDR2 comprises the amino acid sequences of one of SEQ ID NOs: 62-63, the LCDR3 comprises the amino acid sequences of one of SEQ ID NOs: 64-65.

The present disclosure also provides methods of using the anti-ROR1 antibodies comprising three complementarity determining regions (CDRs) on a heavy chain (HCDR1, HCDR2, and HCDR3) and three CDRs on a light chain (LCDR1, LCDR2, and LCDR3), wherein the HCDR1 comprises the amino acid sequences of one of SEQ ID NOs: 66-68, the HCDR2 comprises the amino acid sequences of one of SEQ ID NOs: 69-70, the HCDR3 comprises the amino acid sequences of one of SEQ ID NOs: 71-72, the LCDR1 comprises the amino acid sequences of one of SEQ ID NOs: 73-74, the LCDR2 comprises the amino acid sequences of one of SEQ ID NOs: 75-76, the LCDR3 comprises the amino acid sequences of one of SEQ ID NOs: 77-78.

The present disclosure also provides methods of using the anti-ROR1 antibodies comprising three complementarity determining regions (CDRs) on a heavy chain (HCDR1, HCDR2, and HCDR3) and three CDRs on a light chain (LCDR1, LCDR2, and LCDR3), wherein the HCDR1 comprises the amino acid sequences of one of SEQ ID NOs: 79-81, the HCDR2 comprises the amino acid sequences of one of SEQ ID NOs: 82-83, the HCDR3 comprises the amino acid sequences of one of SEQ ID NOs: 84-85, the LCDR1 comprises the amino acid sequences of one of SEQ ID NOs: 86-87, the LCDR2 comprises the amino acid sequences of one of SEQ ID NOs: 88-89, the LCDR3 comprises the amino acid sequences of one of SEQ ID NOs: 90-91.

The present disclosure also provides methods of using the anti-ROR1 antibodies comprising three complementarity determining regions (CDRs) on a heavy chain (HCDR1, HCDR2, and HCDR3) and three CDRs on a light chain (LCDR1, LCDR2, and LCDR3), wherein the HCDR1 comprises the amino acid sequences of one of SEQ ID NOs: 40-42, the HCDR2 comprises the amino acid sequences of one of SEQ ID NOs: 92-93, the HCDR3 comprises the amino acid sequences of one of SEQ ID NOs: 94-95, the LCDR1 comprises the amino acid sequences of one of SEQ ID NOs: 96-97, the LCDR2 comprises the amino acid sequences of one of SEQ ID NOs: 98-99, the LCDR3 comprises the amino acid sequences of one of SEQ ID NOs: 51-52.

The present disclosure also provides methods of using the anti-ROR1 antibodies comprising a heavy chain variable region and a light chain variable region, wherein the amino acid sequences for the heavy chain variable region and the light chain variable region have the sequences of SEQ ID NOs: 1 and 2.

The present disclosure also provides methods of using the anti-ROR1 antibodies comprising a heavy chain variable region and a light chain variable region, wherein the amino acid sequences for the heavy chain variable region and the light chain variable region have the sequences of SEQ ID NOs: 3 and 4.

The present disclosure also provides methods of using the anti-ROR1 antibodies comprising a heavy chain variable region and a light chain variable region, wherein the amino acid sequences for the heavy chain variable region and the light chain variable region have the sequences of SEQ ID NOs: 5 and 6.

The present disclosure also provides methods of using the anti-ROR1 antibodies comprising a heavy chain variable region and a light chain variable region, wherein the amino acid sequences for the heavy chain variable region and the light chain variable region have the sequences of SEQ ID NOs: 7 and 8.

The present disclosure also provides methods of using the anti-ROR1 antibodies comprising a heavy chain variable region and a light chain variable region, wherein the amino acid sequences for the heavy chain variable region and the light chain variable region have the sequences of SEQ ID NOs: 9 and 10.

The present disclosure also provides methods of using the anti-ROR1 antibodies comprising a heavy chain variable region and a light chain variable region, wherein the amino acid sequences for the heavy chain variable region and the light chain variable region have the sequences of SEQ ID NOs: 11 and 12.

The present disclosure also provides methods of using the anti-ROR1 antibodies comprising a heavy chain variable region and a light chain variable region, wherein the amino acid sequences for the heavy chain variable region and the light chain variable region have the sequences of SEQ ID NOs: 13 and 14.

In some embodiments, the anti-ROR1 antibodies disclosed herein can be used to treat a disease or condition. In some embodiments, the anti-ROR1 antibodies disclosed herein can be used to treat diseases such as cancer. In some embodiments, the anti-ROR1 antibodies disclosed herein can be used as a component of a vaccine. In some embodiments, the anti-ROR1 antibodies disclosed herein can be used as part of an antibody-drug conjugate (ADC). In some embodiments, an anti-ROR1 antibody disclosed herein can be used in methods of treating cancer, for example but not limited to treating non-small-cell lung carcinoma (NSCLC), breast cancer, mesothelioma, pancreatic cancer, renal cancer, prostate cancer, ovarian cancer, or colon cancer.

In some embodiments, the anti-ROR1 antibodies disclosed herein can be used to treat a disease associated with ROR1. In some embodiments, the anti-ROR1 antibodies disclosed herein can be used to treat a disease associated with over-expression of ROR1.

In some embodiments, the anti-ROR1 antibodies disclosed herein comprise cytotoxic activities. In some embodiments, the anti-ROR1 antibodies disclosed herein are cytotoxic to cancer or tumor cells.

In some embodiments, the anti-ROR1 antibodies disclosed herein may be used in a method to a cancer or tumor. In some embodiments, the cancer or tumor comprises a solid cancer or tumor. In some embodiments, the cancer or tumor comprises a non-solid (diffuse) cancer or tumor. In some embodiments, the cancer or tumor comprises a metastasis of a cancer or tumor.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to human or non-human animals to whom treatment with a composition or formulation in accordance with the present anti-ROR1 antibodies is provided. The terms “non-human animals” and “non-human mammals” are used interchangeably herein and include all vertebrates, e.g., mammals, such as non-human primates (e.g., higher primates), sheep, dog, rodent (e.g. mouse or rat), guinea pig, goat, pig, cat, rabbits, cows, horses, or non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In one embodiment, the human is an adult. In another embodiment, the human is a child. The human can be male, female, pregnant, middle-aged, adolescent, or elderly.

Pharmaceutical compositions suitable for use in the methods disclosed herein include compositions wherein the active ingredients are contained in an amount effective to achieve the intended purpose. In one embodiment, a therapeutically effective amount means an amount of active ingredients effective to prevent, alleviate or ameliorate symptoms of disease or prolong the survival of the subject being treated. Determination of a therapeutically effective amount is well within the capability of those skilled in the art.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples (untreated with the composition) can be assigned a relative activity value of 100%.

In one embodiment, the method comprises the step of administering to the subject a composition comprising an effective amount of the anti-ROR1 antibody disclosed herein. In one embodiment, the composition comprises anti-ROR1 antibodies having the heavy chain and light chain CDR sequences as described herein. In another embodiment, the composition comprises anti-ROR1 antibodies having the VH and VL sequences as described herein.

One skilled in the art would appreciate that in some embodiments, modulation of an immune response encompasses a reduction of inflammation or elimination of inflammation in a situation wherein the expected outcome without the use of an anti-ROR1 antibody described herein, would have been inflammation. One skilled in the art would appreciate that in some embodiments, treating a tumor or cancer encompasses a reduction of tumor size, growth, and or spread of the tumor or cancer, compared with the outcome without the use of an anti-ROR1 antibody described herein.

In one embodiment, the present disclosure provides a method of treating a disease in a subject, comprising the step of administering to the subject a composition comprising an effective amount of the anti-ROR1 antibody disclosed herein. In one embodiment, the composition comprises anti-ROR1 antibodies having the heavy chain and light chain CDR sequences as described herein. In another embodiment, the composition comprises anti-ROR1 antibodies having the VH and VL sequences as described herein.

In one embodiment, the present disclosure also provides uses of a composition comprising anti-ROR1 antibodies for treating a disease in a subject. In one embodiment, the composition comprises anti-ROR1 antibodies having the heavy chain and light chain CDR sequences as described herein. In another embodiment, the composition comprises anti-ROR1 antibodies having the VH and VL sequences as described herein.

In one embodiment, the exact amount of the present polypeptides or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

In another embodiment, the disease is a cancer that can be, but is not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

In another embodiment, the disease is an autoimmune disease that can be, but is not limited to, achalasia, amyloidosis, ankylosing spondylitis, anti-gbm/anti-tbm nephritis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, Behcet's disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopenia purpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjögren's syndrome, thrombocytopeniarpura, type 1 diabetes, ulcerative colitis, uveitis, vasculitis, and vitiligo.

In some embodiments, the disease is a transplantation-related diseases such as graft-versus-host disease (GvHD). According to one embodiment, the GVHD is acute GVHD. According to another embodiment, the GVHD is chronic GVHD.

In another embodiment, the present disclosure provides a method of using a polynucleotide to treat a disease or condition as described above, wherein the polynucleotide encodes an anti-ROR1 antibody, or fragments thereof, as described herein.

As used herein, the terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an antibody” or “at least one antibody” may include a plurality of antibodies.

Throughout this application, various embodiments of the present disclosure may be presented in a range format. It should be understood that the description in range format is merely for convenience and brevity and should not be construed as an inflexible limitation on the scope of the anti-ROR1 antibodies and uses thereof. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of ±10% from the indicated number or range of numbers. In another embodiment, the term “about” refers to a deviance of ±5% from the indicated number or range of numbers.

Unless otherwise defined, all technical and/or scientific terms used herein have the same meaning as commonly understood by one of ordinary skill in the art to which the anti-ROR1 antibodies and uses thereof pertains. Although methods and materials similar or equivalent to those described herein can be used in the practice or testing of embodiments of the anti-ROR1 antibodies and uses thereof, methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of making and using the anti-ROR1 antibodies and variations thereof are described. This description is not intended to be limiting and changes in the components, sequence of steps, and other variations would be understood to be within the scope of the present anti-ROR1 antibodies and uses thereof.

It is appreciated that certain features of the anti-ROR1 antibodies and uses thereof, which are, for clarity, described in the context of separate embodiments, may also be provided in combination in a single embodiment. Conversely, various features of the anti-ROR1 antibodies and uses thereof, which are, for brevity, described in the context of a single embodiment, may also be provided separately or in any suitable subcombination or as suitable in any other described embodiment of the anti-ROR1 antibodies and uses thereof. Certain features described in the context of various embodiments are not to be considered essential features of those embodiments, unless the embodiment is inoperative without those elements.

Various embodiments and aspects of the present anti-ROR1 antibodies as delineated hereinabove and as claimed in the claims section below find experimental support in the following example.

### Example 1

### Mouse Hybridoma Monoclonal Antibody Generation

### Methods: Generation of Mouse Hybridoma Anti-ROR1 Antibodies.

Tables 1A and 1B below provide the amino acid sequences of the VH and VL domains, respectively, of anti-ROR1 antibodies disclosed herein. Table 2 below provides the amino acid sequences of the heavy chain and light chain CDR domains.

TABLE 1A
Amino Acid Sequences of Anti-hROR1 VH

| | SEQ ID NOs: | |
|---|---|---|
| Description | (VH) | Amino Acid Sequences |
| mAb004, VH | 1 | EVQLQQSGPELVKPGASVKMSCKASGYTFTDYNIHWV |
| | | KQSHGKSLEWVGYIKPNNGGTTYNQKFKGKATLTVNK |
| | | SSSTAYMELRSLTSEDSAVYYCARRVTVQATGYFDYW |
| | | GQGTTLTVSS |
| mAb006, VH | 3 | EVQLQQSGPELVKPGASVKMSCKASGYTFIDYNIHWVK |
| | | QSHGKSLEWIGYNKPNNGDTRYNEKFKGKATLTVNKSF |
| | | STAYMELRSLTSDDSVVYYCARRGTREGLWDYWGQGT |
| | | TLTVSS |
| mAb008, VH | 5 | EVMLVESGGGLVKPGGSLKLSCAASGFTFSSYTMSWVR |
| | | QTPEKRLEWVASIRGGGDTYYPDSVKGRFTVSRDNAKN |
| | | TLYLQMSSLRSEDTALYYCARHTLYFYDSSHWYFDVW |
| | | GTGTTVAVSS |
| mAb010, VH | 7 | QAQLQQPGAELVKPGASVKLSCKASGYIFISYWMHWV |
| | | KLRPGRGLEWIGWIDPNSYDTKYNEKFKTRATLTVDKP |
| | | SSTAYMQLSSLTSEDSAIYYCARSAEKLGPFFDYWGQG |
| | | TTLTVSS |
| mAb018, VH | 9 | EVQLVESGGDLVKPGGSLKLSCAASGFTFSSYAMSWVR |
| | | QTPDKRLEWVATISSGVSYTYYPDSVKGRFTISRDNAKN |
| | | TLYLQMSSLKSEDTAMYYCTRRAKNDYGCFPYWGRGT |
| | | LVTVSA |
| mAb019, VH | 11 | QAYLQQSGAELVRPGASVKMSCKASGYTFTSYNMHWV |
| | | KQTPRQGLEWIGAIYLGNGDTSYNQKFKGKATLTVDKS |
| | | SSTVYMQLSSLTSEDSAVYFCARRGKGGYYAMDYWGQ |
| | | GTSVTVSS |
| mAb020, VH | 13 | EVMLVESGGGLVKPGGSLKLSCAASGFTFSSYTMSWVR |
| | | QTPEKRLEWVAAIRGGGGNTYYADSVKGRFTISRDNAK |
| | | NTLYLQMSSLRSEDTALYYCTRHTLYYYDSSHWYFDV |
| | | WGTGTTVTVSS |

TABLE 1B
Amino Acid Sequences of Anti-hROR1 VL

| | SEQ ID NOs: | |
|---|---|---|
| Description | (VL) | Amino Acid Sequences |
| mAb004, VL | 2 | DVLMTQTPLSLPVSLGDHASISCRSSQSIVHLNGNTYLE |
| | | WYLQKPGQSPKLLIFKVSNRFSGVPDRFSGSGSGTDFTL |
| | | KISRVEAEDLGIYYCFQGSLFPYTFGGGTKLEIK |
| mAb006, VL | 4 | DVLMTQTPLSLPVSLGDQVSISCRSSLSLLHINGNTYLE |
| | | WYLQKPGQSPKLLIYKASNRFSGVPDRFSGSGSGTDFTL |
| | | KISRVEAEDLGIYYCFQGSRFPYTFGGGTKLEIK |
| mAb008, VL | 6 | DIKMTQSPSSMYASLGERATITCKASQDINNYLSWFQQ |
| | | KPGKSPKTLIYRADRLVDGVPSRFSGSGSGQDYSLTISSL |
| | | EFEDMGIYYCLQYDEFPWTFGGGSKLEIK |
| mAb010, VL | 8 | DVQITQSPSYLAASPGETISINCRASKSISKYLAWYQEKP |
| | | GKSNKLLIYSGSTLPSGIPSRFSGSGFGTDFTLTISGLEPE |
| | | DFAMYYCQQHNEYPWTFGGGTKLEIK |
| mAb018, VL | 10 | DIVMTQSPSSLAMSVGQKVTMSCKSSQSLLNSSNQKNY |
| | | LAWYQQKPGQSPKLLVYFASTRESGVPDRFIGSGSGTYF |
| | | TLTISSVQAEDLADYFCQQHYSTPRTFGGGTKLEIK |
| mAb019, VL | 12 | DVLMTQTPLSLPVSLGDQASISCRSSQSIVHINGNTYLE |
| | | WYLQKPGQSPKLLIYKVSNRFSGVPDRFSGSGSGTDFTL |
| | | KITRLEAEDLGVYYCFQGSLFPYTFGGGTKLEIK |
| mAb020, VL | 14 | DIKMTQSPSSMYASLGERVTITCKASQDINSYLSWFQQK |
| | | PGKSPKTLIYRANRLVDGVPSRFSGSGSGQDYSLTISSLE |
| | | YEDMGIYYCLQYDEFPWTFGGGTKLEIK |

TABLE 2
Amino Acid Sequences of Anti-hROR1 CDRs

| | | SEQ ID | |
|---|---|---|---|
| Antibody | Description | NOS | Amino Acid Sequences |
| mAb004 | HCDR1 | 15 | DYNIH |
| | | 16 | YTFTDYNIH |
| | | 17 | GYTFTDYNIH |
| | HCDR2 | 18 | YIKPNNGGTTYNQKFKG |
| | | 19 | WVGYIKPNNGGTTY |
| | HCDR3 | 20 | RVTVQATGYFDY |
| | | 21 | RRVTVQATGYFDY |
| | LCDR1 | 22 | RSSQSIVHLNGNTYLE |
| | | 23 | QSIVHLNGNTYLE |
| | LCDR2 | 24 | KVSNRFS |
| | | 25 | LLIFKVSNRFS |
| | LCDR3 | 26 | FQGSLFPYT |
| | | 27 | FQGSLFPY |
| mAb006 | HCDR1 | 15 | DYNIH |
| | | 28 | YTFIDYNIH |
| | | 29 | GYTFIDYNIH |
| | HCDR2 | 30 | YNKPNNGDTRYNEKFKG |
| | | 31 | WIGYNKPNNGDTRY |
| | HCDR3 | 32 | RGTREGLWDY |
| | | 33 | RRGTREGLWDY |
| | LCDR1 | 34 | RSSLSLLHINGNTYLE |
| | | 35 | LSLLHINGNTYLE |
| | LCDR2 | 36 | KASNRFS |
| | | 37 | LLIYKASNRFS |
| | LCDR3 | 38 | FQGSRFPYT |
| | | 39 | FQGSRFPY |
| mAb008 | HCDR1 | 40 | SYTMS |
| | | 41 | FTFSSYTMS |
| | | 42 | GFTFSSYTMS |
| | HCDR2 | 43 | SIRGGGDTYYPDSVKG |
| | | 44 | WVASIRGGGDTYY |
| | HCDR3 | 45 | HTLYFYDSSHWYFDV |
| | | 46 | RHTLYFYDSSHWYFDV |
| | LCDR1 | 47 | KASQDINNYLS |
| | | 48 | QDINNYLS |
| | LCDR2 | 49 | RADRLVD |
| | | 50 | TLIYRADRLVD |
| | LCDR3 | 51 | LQYDEFPWT |
| | | 52 | LQYDEFPW |
| mAb010 | HCDR1 | 53 | SYWMH |
| | | 54 | YIFISYWMH |
| | | 55 | GYIFISYWMH |
| | HCDR2 | 56 | WIDPNSYDTKYNEKFKT |
| | | 57 | WIGWIDPNSYDTKY |
| | HCDR3 | 58 | SAEKLGPFFDY |
| | | 59 | RSAEKLGPFFDY |
| | LCDR1 | 60 | RASKSISKYLA |
| | | 61 | KSISKYLA |
| | LCDR2 | 62 | SGSTLPS |
| | | 63 | KLLIYSGSTLPS |
| | LCDR3 | 64 | QQHNEYPWT |
| | | 65 | QQHNEYPW |
| mAb018 | HCDR1 | 66 | SYAMS |
| | | 67 | FTFSSYAMS |
| | | 68 | GFTFSSYAMS |
| | HCDR2 | 69 | TISSGVSYTYYPDSVKG |
| | | 70 | WVATISSGVSYTYY |
| | HCDR3 | 71 | RAKNDYGCFPY |
| | | 72 | TRRAKNDYGCFPY |
| | LCDR1 | 73 | KSSQSLLNSSNQKNYLA |
| | | 74 | QSLLNSSNQKNYLA |
| | LCDR2 | 75 | FASTRES |
| | | 76 | LLVYFASTRES |
| | LCDR3 | 77 | QQHYSTPRT |
| | | 78 | QQHYSTPR |
| mAb019 | HCDR1 | 79 | SYNMH |
| | | 80 | YTFTSYNMH |
| | | 81 | GYTFTSYNMH |
| | HCDR2 | 82 | AIYLGNGDTSYNQKFKG |
| | | 83 | WIGAIYLGNGDTSY |
| | HCDR3 | 84 | RGKGGYYAMDY |
| | | 85 | RRGKGGYYAMDY |
| | LCDR1 | 86 | RSSQSIVHINGNTYLE |
| | | 87 | QSIVHINGNTYLE |
| | LCDR2 | 88 | KVSNRFS |
| | | 89 | LLIYKVSNRFS |
| | LCDR3 | 90 | FQGSLFPYT |
| | | 91 | FQGSLFPY |
| mAb020 | HCDR1 | 40 | SYTMS |
| | | 41 | FTFSSYTMS |
| | | 42 | GFTFSSYTMS |
| | HCDR2 | 92 | AIRGGGGNTYYADSVKG |
| | | 93 | WVAAIRGGGGNTYYA |
| | HCDR3 | 94 | HTLYYYDSSHWYFDV |
| | | 95 | RHTLYYYDSSHWYFDV |
| | LCDR1 | 96 | KASQDINSYLS |
| | | 97 | QDINSYLS |
| | LCDR2 | 98 | RANRLVD |
| | | 99 | TLIYRANRLVD |
| | LCDR3 | 51 | LQYDEFPWT |
| | | 52 | LQYDEFPW |

1. An isolated anti-ROR1 antibody comprising three complementarity determining regions (CDRs) on a heavy chain (HCDR1, HCDR2, and HCDR3) and three CDRs on a light chain (LCDR1, LCDR2, and LCDR3), wherein
(i) the HCDR1 comprises the amino acid sequences of one of SEQ ID NOs: 15-17, the HCDR2 comprises the amino acid sequences of one of SEQ ID NOs: 18-19, the HCDR3 comprises the amino acid sequences of one of SEQ ID NOs: 20-21, the LCDR1 comprises the amino acid sequences of one of SEQ ID NOs: 22-23, the LCDR2 comprises the amino acid sequences of one of SEQ ID NOs: 24-25, the LCDR3 comprises the amino acid sequences of one of SEQ ID NOs: 26-27; or
(ii) the HCDR1 comprises the amino acid sequences of one of SEQ ID NOs: 15, 28, 29, the HCDR2 comprises the amino acid sequences of one of SEQ ID NOs: 30-31, the HCDR3 comprises the amino acid sequences of one of SEQ ID NOs: 32-33, the LCDR1 comprises the amino acid sequences of one of SEQ ID NOs: 34-35, the LCDR2 comprises the amino acid sequences of one of SEQ ID NOs: 36-37, the LCDR3 comprises the amino acid sequences of one of SEQ ID NOs: 38-39; or
(iii) the HCDR1 comprises the amino acid sequences of one of SEQ ID NOs: 40-42, the HCDR2 comprises the amino acid sequences of one of SEQ ID NOs: 43-44, the HCDR3 comprises the amino acid sequences of one of SEQ ID NOs: 45-46, the LCDR1 comprises the amino acid sequences of one of SEQ ID NOs: 47-48, the LCDR2 comprises the amino acid sequences of one of SEQ ID NOs: 49-50, the LCDR3 comprises the amino acid sequences of one of SEQ ID NOs: 51-52; or
(iv) the HCDR1 comprises the amino acid sequences of one of SEQ ID NOs: 53-55, the HCDR2 comprises the amino acid sequences of one of SEQ ID NOs: 56-57, the HCDR3 comprises the amino acid sequences of one of SEQ ID NOs: 58-59, the LCDR1 comprises the amino acid sequences of one of SEQ ID NOs: 60-61, the LCDR2 comprises the amino acid sequences of one of SEQ ID NOs: 62-63, the LCDR3 comprises the amino acid sequences of one of SEQ ID NOs: 64-65; or
(v) the HCDR1 comprises the amino acid sequences of one of SEQ ID NOs: 66-68, the HCDR2 comprises the amino acid sequences of one of SEQ ID NOs: 69-70, the HCDR3 comprises the amino acid sequences of one of SEQ ID NOs: 71-72, the LCDR1 comprises the amino acid sequences of one of SEQ ID NOs: 73-74, the LCDR2 comprises the amino acid sequences of one of SEQ ID NOs: 75-76, the LCDR3 comprises the amino acid sequences of one of SEQ ID NOs: 77-78; or
(vi) the HCDR1 comprises the amino acid sequences of one of SEQ ID NOs: 79-81, the HCDR2 comprises the amino acid sequences of one of SEQ ID NOs: 82-83, the HCDR3 comprises the amino acid sequences of one of SEQ ID NOs: 84-85, the LCDR1 comprises the amino acid sequences of one of SEQ ID NOs: 86-87, the LCDR2 comprises the amino acid sequences of one of SEQ ID NOs: 88-89, the LCDR3 comprises the amino acid sequences of one of SEQ ID NOs: 90-91; or
(vii) the HCDR1 comprises the amino acid sequences of one of SEQ ID NOs: 40-42, the HCDR2 comprises the amino acid sequences of one of SEQ ID NOs: 92-93, the HCDR3 comprises the amino acid sequences of one of SEQ ID NOs: 94-95, the LCDR1 comprises the amino acid sequences of one of SEQ ID NOs: 96-97, the LCDR2 comprises the amino acid sequences of one of SEQ ID NOs: 98-99, the LCDR3 comprises the amino acid sequences of one of SEQ ID NOs: 51-52.

2. The anti-ROR1 antibody of claim 1, wherein the antibody comprises a heavy chain variable region and a light chain variable region, said heavy chain variable region and light chain variable region comprise the amino acid sequences of SEQ ID NOs: 1 and 2; SEQ ID NOs: 3 and 4; SEQ ID NOs: 5 and 6; SEQ ID NOs: 7 and 8; SEQ ID NOs: 9 and 10; SEQ ID NOs: 11 and 12; or SEQ ID NOs: 13 and 14.

3. The anti-ROR1 antibody of claim 1, wherein the antibody is an IgG, a Fv, a scFv, a Fab, a F(ab′)₂, a minibody, a diabody, a triabody, a nanobody, a bispecific antibody, a tri-specific antibody, a multi-specific antibody, or a single domain antibody.

4. The anti-ROR1 antibody of claim 3, wherein said IgG is IgG1, IgG2, IgG3, or IgG4.

5. A composition comprising the anti-ROR1 antibody of claim 1 and a pharmaceutically acceptable carrier.

6. A nucleic acid construct comprising one or more nucleic acid sequences, said nucleic acid sequences encode a light chain, or a heavy chain, or fragments thereof of the anti-ROR1 antibody of claim 1.

7. An expression vector comprising the nucleic acid construct of claim 6.

8. A host cell comprising the expression vector of claim 7.

9. A method of treating a disease in a subject, comprising the step of administering to the subject a composition comprising an effective amount of the anti-ROR1 antibody of claim 1.

10. The method of claim 9, wherein the disease is a cancer, an autoimmune disease, GvHD, a viral infection, or a bacterial infection.

11. The method of claim 10, wherein the cancer comprises a solid cancer or a non-solid (diffuse) cancer.

12. The method of claim 10, wherein the cancer comprises a metastasis of a cancer.
